(19) United States
(12) Patent Application Publication
(10) Pub. No.: US 2010/0074958 A1
(43) Pub. Date: Mar. 25, 2010

(54) METHODS AND COMPOSITIONS FOR TARGETING FENESTRATED VASCULATURE

(75) Inventors: Paolo Decuzzi, Bari (IT); Mauro Ferrari, Houston, TX (US)

Correspondence Address: FOLEY AND LARDNER LLP;SUITE 500, 3000 K STREET NW, WASHINGTON, DC, 20007, US

(73) Assignees: Board of Regents of the University of Texas System; The Ohio State University Research Foundation

(21) Appl. No.: 12/443,230

(22) PCT Filed: Sep. 28, 2006
(86) PCT No.: PCT/US06/38196

§ 371 Date: Mar. 27, 2009

(51) Int. Cl. A61K 9/14; A61K 38/43; A61K 31/337; A61K 31/704; A61P 9/00; A61P 7/04

(52) U.S. Cl. 424/489; 424/94.1; 514/449; 514/34

(57) ABSTRACT

Targeting of a fenestrated vasculature at a body site by micro or nanoparticles can be increased by using particles that have a radius substantially equal to a critical radius of a normal vasculature at the body site. The particles can be used for treating or monitoring a physiological condition responsible for the fenestrated vasculature. A method of improving an ability of micro or nanoparticles to target fenestrated blood vessels in a body site by selecting particles from a population of the micro or nanoparticles, where the selected particles have a radius that permits enhanced delivery into the fenestrated blood vessels.

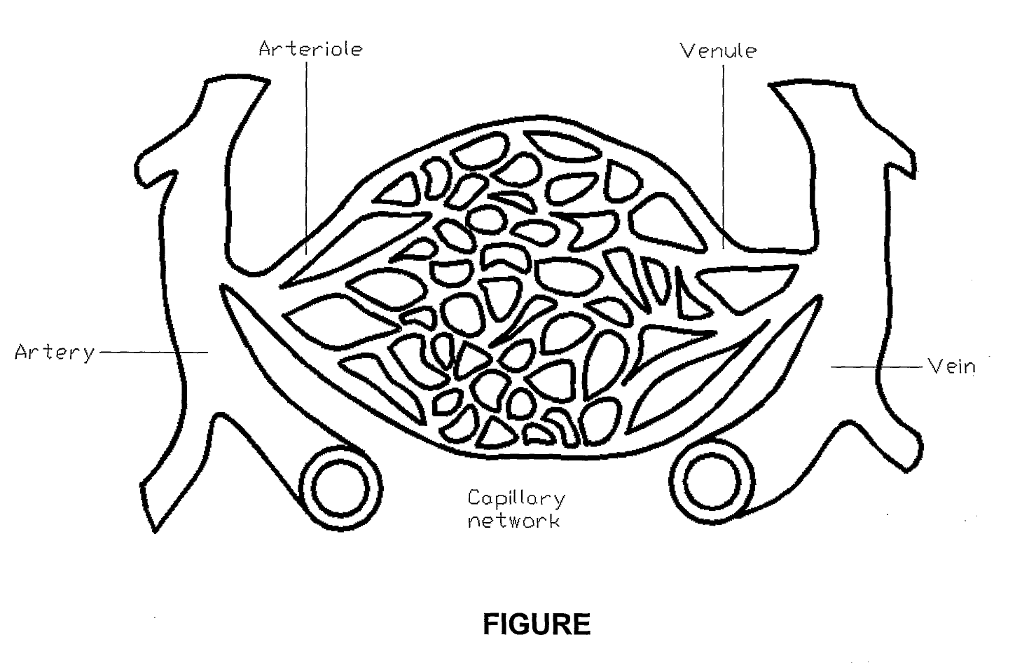

### BACKGROUND

1. Field of the Invention

The present application relates generally to compositions and methods utilizing micro and nanoparticles for delivery of active agents, such as therapeutic or imaging agents, and more particularly to compositions and methods utilizing micro and nanoparticles for targeting fenestrated endothelium of blood vessels and for treating and monitoring a physiological condition responsible for the fenestrated endothelium.

2. Description of Related Art

Nano and microscale particles, also known as nanovectors, can be used for delivery of active agents, such as therapeutic or imaging agents, see e.g. Ferrari, M. Nat. Rev. Cancer 5:161, 2005. Illustrative examples of such nanovectors include silicon particles, see, e.g., Cohen, M. H., K. Melnik, A. Boiasrki, M. Ferrari, F. J. Martin. Biomed. Microdevices 5:253-259, 2003, polymer-based particles, see, e.g., Duncan, Nat. Rev./Drug Discov. 2:347-360, 2003; quantum dots, see e.g. Alivisatos, P. Science 271:933-937, 1996; iron oxide particles, see, e.g., Winter, P. M., S. A. Wickline, and G. M. Lanza. Cancer Res. 63:5838-5843, 2003; gadolinium-containing particles, see e.g. Oyewumi, M. O., and R. J. Mumper. J. Control. Release 24:613-626, 2004; gold nanoshells, see, e.g., Goldsmith, H. L., and V. T. Turitto. Thromb. Haemost. 55:415-435, 1986 and low-density lipid particulates, see, e.g., Bloch, S. H., M. Wan, P. A. Dayton, and K. W. Ferrara. Appl. Phys. Lett. 84(4):631-633, 2004.

Physiological conditions such as tumor or inflammation can result in formation of fenestrations in endothelium of blood vessels. To treat or monitor such physiological conditions micro or nanoparticles should be able to reach the blood vessels affected by the fenestrations. Accordingly, a need exists to develop particles that can effectively target the fenestrated blood vessels, i.e., blood vessels having fenestrations in their endothelium due to a physiological condition such as tumor or inflammation.

### SUMMARY

In accordance with certain embodiments of the present invention, a method is provided for targeting fenestrated blood vessels in a body site, comprising administering to a subject in need thereof a composition comprising particles having a radius that permits enhanced delivery into fenestrated blood vessels of the body site, wherein the particles comprise at least one active agent. In some embodiments, the at least one active agent comprises a therapeutic agent. In some embodiments, the therapeutic agent is an anticancer agent. In some embodiments, the active agent comprises an imaging agent.

In some embodiments, the particles comprise a nanoporous material, such as, for example, a nanoporous silicon, a nanoporous oxide material, or a nanoporous silicon dioxide.

In certain other embodiments, the particles comprise a biodegradable material.

In some embodiments, the particles comprise at least one recognition moiety disposed on a surface of each of the particles. In certain embodiments, the at least one recognition moiety comprises a renormalized vasculature recognition moiety. In certain embodiments, the at least one recognition moiety comprises a coopted vasculature recognition moiety. In certain embodiments, the at least one recognition moiety comprises an angiogenesis vasculature recognition moiety. In certain embodiments, a recognition moiety comprises hydrophilic polymer chains.

In some embodiments, one or more of said particles are selected from the group consisting of liposomes, fullerene nanoparticles, semiconductor nanoparticles and metal nanoparticles.

In some embodiments, an above-described method comprises fabricating one or more of said particles. For example, such fabricating may comprise fabricating by a top-down technique.

In some embodiments of an above-described method, administering comprises injecting the composition intravascularly into the subject, e.g., in a vasculature of the body site.

In certain embodiments of an above-described method, the subject is a mammal, preferably a human. The body site may be brain, skin, skeletal muscle, lung, heart, kidney, stomach, or intestine, for example.

The composition employed in an above-described method comprises a suspension of the particles, in some embodiments.

A condition responsible for the fenestrated blood vessel condition may be a tumor, for instance, and in some embodiments of an above-described method, the method includes administering to the subject a vasculature normalizing agent.

Also provided in accordance with certain embodiments of the present invention is a method of improving efficacy of a composition that comprises particles that contain at least one active agent. This method comprises selecting a second population of particles from a first population of micro or nanoparticles, such that the particles in the second population have a radius that permits enhanced delivery into fenestrated blood vessels of a target body site. The method further comprises forming a composition comprising the selected second population of particles. In certain embodiments, the selected particles constitute at least 10% of the first particles by number. In certain embodiments, the selected particles constitute at least 20% of the first particles by number. In certain embodiments, the selected particles constitute at least 50% of the first particles by number. In certain embodiments, the selected particles constitute at least 80% of the first particles by number.

Still other embodiments of the present invention provide a method of improving the ability of micro or nanoparticles to target fenestrated blood vessels in a body site by selecting particles from a population of the micro or nanoparticles, where the selected particles have a radius that facilitates or permits enhanced delivery into the fenestrated blood vessels. These and other embodiments, features and advantages will be apparent from the following description and drawings.

### BRIEF DESCRIPTION OF THE DRAWINGS

The FIGURE schematically depicts blood vessels of the circulatory system.

### DETAILED DESCRIPTION

### Definitions

Unless otherwise specified “a” or “an” means one or more.

The term “critical radius” refers to a critical radius of a normal non-fenestrated blood vessel, at a particular body site, which is defined below.

“Recognition moiety” can be any factor that can facilitate targeting of a specific site by a particle. The recognition moiety can be a chemical targeting moiety, a physical targeting moiety or a combination thereof. The chemical targeting moiety can be a chemical group or molecule on a surface of the particle; the physical targeting moiety can be a specific physical property of the particle such as a surface such or hydrophobicity.

“Microparticle” means a particle having a maximum characteristic size from 1 micron to 1000 microns, or from 1 micron to 100 microns. “Nanoparticle” means a particle having a maximum characteristic size of less than 1 micron.

“Body site” means a part of a body of a subject that has blood vessels. Body site can be an organ in a body of a subject such as brain, heart, kidney, skin, lung, muscle, stomach or intestine.

“Fenestrated blood vessel” refers to a blood vessel that has a fenestrated endothelium. caused by a physiological condition, such as a tumor or inflammation, but the term “fenestrated blood vessel” excludes blood vessels that normally have a fenestrated endothelium such as, for example, capillaries of the gastrointestinal tract, capillaries of glomerulus in kidney or sinusoids in the bone marrow. Fenestrated blood vessel further means a blood vessel having a hydraulic permeability higher than a normal blood vessel. Characteristic values of hydraulic permeabilities Lₚ for capillaries in selected body sites in a human body are presented, for example, in Ganong, W. F. Review of Medical Physiology, 21st ed. New York: Lange Medical Books/McGraw-Hill, Medical Publishing Division, 2003, as summarized in Table 1.

TABLE 1

| | Hydraulic permeability |
|---|---|
| Organ | Lₚ, μm/Pa s |
| Brain | 3 |
| Skin | 100 |
| Skeletal muscle | 250 |
| Lung | 340 |
| Heart | 860 |

The present disclosure incorporates herein by reference in its entirety P. Decuzzi, F. Causa, M. Ferrari and P. A. Netti “The Effective dispersion of Nanovectors Within the Tumor Microvasculature,” Annals of Biomedical Engineering, 34(4): 633-641, 2006.

The inventors have recognized that an effective diffusivity of fenestrated blood vessels, such as fenestrated capillaries, is lower than an effective diffusivity of normal blood vessels, such as normal capillaries, at the same body site. Thus, for a given size, particles, such as nanovectors, tend to reach the normal blood vessels in larger numbers than the fenestrated blood vessels.

The inventors have also recognized that, for a particular blood vessel, there is a critical radius of a particle, for which an effective diffusivity of particles has a minimum. Such a critical radius can exist for both fenestrated blood vessels and normal blood vessels. The critical radius for normal vessels is usually smaller than the critical radius for fenestrated vessels at the same body site.

The inventors have determined that administration of particles having a radius that is substantially equal to a critical radius for a normal blood vessel at a body site can increase a number of particles reaching a fenestrated blood vessel at the body site.

Accordingly, in one embodiment, the invention provides a method of targeting one or more fenestrated blood vessels in a body site of a subject, preferably a mammal and more preferably a human, in need thereof by administering to the subject a composition comprising particles with a radius substantially equal to a critical radius for the body site, wherein the particles comprise at least one active agent. The composition can also comprise additional particles, i.e., particles that do not have a radius substantially equal to the critical radius for the body site. In various embodiments, the particles with a radius substantially equal to the critical radius for the body site can constitute at least 10%, or at least 25%, or at least 50%, or at least 75%, or at least 90% by number of all the particles in the composition.

In one embodiment, the at least one active agent comprises a therapeutic agent and thus the method of targeting a fenestrated vasculature is used for treating a physiological condition responsible for the fenestrated vasculature. In another embodiment, the at least one active agent comprises an imaging agent and the method of targeting a fenestrated vasculature can be used for monitoring a physiological condition responsible for the fenestrated vasculature. In still another embodiment, the at least one active agent comprises both an imaging agent and a therapeutic agent and the method of targeting a fenestrated vasculature is used for both monitoring and treating a physiological condition responsible for the fenestrated vasculature.

In another embodiment, the invention provides a method of improving targeting of particles to fenestrated vasculature in a body site by selecting particles from a population of the particles according to their size (e.g., radius) such that a relative amount of the selected particles having a size that permits enhanced delivery to the fenestrated vasculature of the body site. The selected particles preferably have a radius substantially equal to a critical radius for the body site. Selecting of the particles according to their size can be performed, for example, using Zetasizer™ Nano series instrument from Malvern Instruments, Worcestershire, United Kingdom. The relative amount of the selected particles is preferably at least 10%, or at least 25%, or at least 50%, or at least 75%, or at least 90% by number in the final product. Upon selecting the particles according to their size, a composition is formed from the selected particles. Such a composition is more effective for targeting the fenestrated vasculature at the body site than a composition formed from the starting population of the particles. When the particles comprise a therapeutic agent, the composition with an increased relative amount of the selected particles can reduce the overall dosage of the therapeutic agent in the composition for effective treatment of a physiological condition responsible for the fenestrated vasculature at the body side compared to an unselected composition, i.e., a composition formed from an unselected population of the particles. Similarly, when the particles comprise an imaging agent, the composition enriched with the selected particles can reduce the amount of the particles for monitoring a physiological condition responsible for the fenestrated vasculature at the body site compared to the unselected composition.

### Critical Radius

Preferably, a critical radius for a normal non-fenestrated blood vessel is determined using the following formula:

$${a}_{cr} = \frac{2}{π \sqrt{3}} \frac{{k}_{B} T}{η RU}$$

where k_{B} is Boltzmann constant, T is an absolute temperature of the blood expressed in Kelvins; R is a radius of the blood vessel; U is a blood velocity in the blood vessel, η is a viscosity of the blood.

For the blood viscosity one preferably uses an average value of 10⁻³ Pa s for a human, or alternatively, one can determine a value of the blood viscosity experimentally. For example, a value of the blood viscosity is evaluated from a plasma viscosity determined with a glass capillary viscometer, hematocrit and mean wall share rate as disclosed in Weaver J. P. et al. Clin. Sci. 36: 1-10, 1969 and Dammers R., et al. J. Appl. Physiol. 94:485-489, 2003, which are both incorporated herein by reference in their entirety.

For the blood velocity in the blood vessel and the radius of the blood vessel, one preferably uses values summarized in Table 2 as taken from Ganong, W. F. Review of Medical Physiology, 21st ed. New York: Lange Medical Books/McGraw-Hill Medical Publishing Division, 2003, incorporated herein by reference in its entirety. The FIGURE schematically depicts blood vessels of the circulatory system.

TABLE 2

| Blood vessel | U, μm/s | Rₑ, μm |
|---|---|---|
| Aorta | 4 × 10⁵ | 25 × 10³ |
| Artery | 1 × 10⁵ | 4 × 10³ |
| Arteriole | 5 × 10³ | 20-50 |
| Capillary | 10-100 | 5-10 |
| Venules | 5 × 10² | 20-50 |
| Vein | 5 × 10⁴ | 2-5 × 10³ |
| Vena cava | 1 × 10⁵ | 30 × 10³ |

Alternatively, one can determine the blood velocity and the radius of the blood vessel experimentally using, for example, an ultrasound system, such as Ultramark 9 plus™, Advanced

Technology Laboratories, Bellevue, Wash. as detailed in Dammers R., et al. J. Appl. Physiol. 94:485-489, 2003. In this case, the blood velocity can be a mean center velocity averaged over a heart cycle.

Because, for a particular body site, there can be a plurality of blood vessels of the same type, one can use values of the blood velocity and the radius averaged over such blood vessels, when calculating the critical radius for the body site.

As the fenestrations associated with a physiological condition, such as a tumor or inflammation, usually affect smaller blood vessels of the body site, such as capillaries, the critical radius for the body site is usually a critical radius for normal capillaries of the body site. A critical radius can be calculated for any type of a blood vessel, however.

When estimating a critical radius for a body site, one can optionally further take into account permeability of blood vessels as follows:

$${a}_{cr} = \frac{2}{π \sqrt{3}} \frac{{k}_{B} T}{η RU} {\int }_{0}^{1} \frac{d \tilde{z}}{f ( Ω , Π , \tilde{z} )} , \\ where$$

$$f ( Ω , Π , \tilde{z} ) = {[ \frac{( 1 + {e}^{2 \tilde{z} Π} ) - {e}^{Π} ( 1 + {e}^{2 ( \tilde{z} - 1 ) Π} ) Ω}{2 - {e}^{Π} ( 1 + {e}^{- 2 Π} ) Ω} {e}^{- Π \tilde{z}} ]}^{2}$$

$$or$$

$$f ( Ω , Π , \tilde{z} ) = {[ 1 + \tilde{z} [ 2 Ω + ( 1 - Ω ) \tilde{z} ] \frac{{Π}^{2}}{Ω - 1} ]}^{- 1} ,$$

In the above formula,

$$Π = \frac{4 l}{R} \sqrt{\frac{η}{R} {L}_{p}} , Ω = \frac{{p}_{art} - {π}_{int}}{{p}_{ven} - {π}_{int}} ,$$

where l is a length of a blood vessel such as a capillary; pₐᵣₜ, even and πᵢₙₜ are respectively a blood pressure at the arterial side of capillary, a blood pressure at the venous side of capillary and interstitial fluid pressure in the capillary. The integration in the above formula can be performed either analytically or numerically using, for example, standard software programs.

Lₚ is a permeability of a blood vessel, see, e.g., Table 1.

l, an average length of capillaries for a particular body site can be obtained using histological inspections as known to those of ordinary skill in the art.

pₐᵣₜ and pᵥₑₙ, blood pressures at the arterial and venous sides of capillaries for a particular body site, can be obtained, for example, using catherers immersed in corresponding arterial and venous blood vessels fluidically connected to the capillaries at the body site. πᵢₙₜ, an interstitial fluid pressure, can be typically considered to be zero for normal capillaries. Still, if necessary, the interstitial fluid pressure can be measured for blood vessels using methods described in Boucher, Y., Baxter, L. T., and Jain, R. K. (1990) Interstitial Pressure Gradient in tissue-isolated and subcutaneous tumore: Implications for therapy. Cancer Res. 50, 4478-4484, incorporated herein by reference in its entirety.

Because values of permeability Lₚ can change significantly from one body site to another, see, e.g., Table 1, a critical radius can also vary from organ to organ. Thus, for example, selected particles for targeting the brain can be smaller in radius than selected particles delivered to the heart.

### Particles

A selected particle means a particle optimized for targeting a fenestrated microvasculature, i.e. one or more fenestrated blood vessels, of a particular body site. In other words, the selected particles have a radius substantially equal to a critical radius of the body site. “Substantially equal” means that the radius of the selected particle equals the critical radius within a certain margin. Such a margin can be determined, for example, from variations of radii and blood velocities of normal blood vessels in the body site. In some embodiments, the margin can be, for example, 30% or less, i.e., the radius of the selected particle is from 0.7 to 1.3 of the critical radius. In various embodiments, the margin can be 20% or less, or 10% or less, or 5% or less, or 3% or less, or 1% or less.

A selected particle can be a micro or nanoparticle of any type. For example, the selected particle can be a liposome, a polymer-based particle, a silicon-and silica based particle, a quantum dot, a gold nanoshell or a dendrimer.

Selected particles can be fabricated to have a specific radius or alternatively selected particles can be screened from a pool of particles having a distribution of sizes. The selection from the pool of particles can be performed, for example, using Zetasizer™ Nano series instrument from Malvern Instruments, Worcestershire, United Kingdom, which allows measuring radii of the particles.

The selected particle can be fabricated by any suitable Method. Preferably the fabrication method provides a control over the size of the particle. For example, in some embodiments, the particles are fabricated by a top-down microfabrication or nanofabrication methods such as photolithography, electron beam lithography, X-ray lithography, deep UV lithography or nanoprint lithography. The advantage of using the top-down fabrication methods can be that such methods provide for a scaled up production of particles that are uniform in dimensions.

In some embodiments, selected particles can have on their surfaces targeting moieties such as ligands, aptamers or antibodies. For example, ligands can be chemically linked to appropriate reactive groups on the surface of the particles. Protein ligands can be linked to amino- and thiol-reactive groups under conditions effective to form thioether or amide bonds respectively. Methods for attaching antibody or other polymer binding agents to an inorganic or polymeric support are detailed, for example, in Taylor, R., Ed., Protein Immobilization Fundamentals and Applications, pp. 109110 (1991).

In some embodiments, the selected particle can have one or more channels connecting a reservoir with the surface. In some embodiments, the reservoir and the channels can be pores in the body of the particle. In such case, the particle can comprise a porous or nanoporous material. The pores of the porous or nanoporous material can be controlled to achieve a desired load of the active agent and a desired release rate. The nanoporous material with controllable pore size can be an oxide material, such as SiO₂, Al₂O₃, or TiO₂. Fabrication of nanoporous oxide particles, also known as sol gel particles, is detailed, for example, in Paik J. A. et. al. J. Mater. Res., Vol. 17, August 2002. The nanoporous material with controllable pore size can be also nanoporous silicon. For details of fabrication of nanoporous silicon particles, see Cohen M. H. et. al. Biomedical Microdevices 5:3, 253-259, 2003.

In some other embodiments, the selected particle has no channels at all. Such particle can comprise, for example, a biodegradable material. For example, the particle may be formed of metals such as iron, titanium, gold, silver, platinum, copper, and alloys and oxides thereof. The biodegradable material can be also a biodegradable polymer such as polyorthoesters, polyanhydrides, polyamides, polyalkylcyanoacrylates, polyphosphazenes, and polyesters. Exemplary biodegradable polymers are described, for example, in U.S. Pat. Nos. 4,933,185, 4,888,176, and 5,010,167. Specific examples of such biodegradable polymer materials include poly(lactic acid), polyglycolic acid, polycaprolactone, polyhydroxybutyrate, poly(N-palmitoyl-trans-4-hydroxy-L-proline ester) and poly(DTH carbonate).

In some embodiments, the particle is an active agent per se.

### Active Agent

The active agent can be a therapeutic compound or an imaging agent, for example. The selection of the active agent depends on the desired application. The therapeutic agent may be any physiologically or pharmacologically active substance that can produce a desired biological effect in fenestrated vasculature of the subject, such as a mammal or a human. The therapeutic agent may be any inorganic or organic compound, without limitation, including peptides, proteins, nucleic acids, and small molecules. The therapeutic agent may be in various forms, such as an unchanged molecule, molecular complex, pharmacologically acceptable salt, such as hydrochloride, hydrobromide, sulfate, laurate, palmitate, phosphate, nitrite, nitrate, borate, acetate, maleate, tartrate, oleate, salicylate, and the like. For acidic therapeutic agent, salts of metals, amines or organic cations, for example, quaternary ammonium, can be used. Derivatives of drugs, such as bases, esters and amides also can be used as a therapeutic agent. A therapeutic agent that is water insoluble can be used in a form that is a water soluble derivative thereof, or as a base derivative thereof, which in either instance, or by its delivery, is converted by enzymes, hydrolyzed by the body pH, or by other metabolic processes to the original therapeutically active form.

The therapeutic agent can be a chemotherapeutic agent, an immunosuppressive agent, a cytokine, a cytotoxic agent, a nucleolytic compound, a radioactive isotope, a receptor, and a pro-drug activating enzyme, which may be naturally occurring or produced by synthetic or recombinant methods, or any combination thereof.

Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) can have particular utility as the therapeutic agent.

A cancer chemotherapy agent is a preferred therapeutic agent. Useful cancer chemotherapy drugs include nitrogen mustards, nitrosorueas, ethyleneimine, alkane sulfonates, tetrazine, platinum compounds, pyrimidine analogs, purine analogs, antimetabolites, folate analogs, anthracyclines, taxanes, vinca alkaloids, topoisomerase inhibitors and hormonal agents. Exemplary chemotherapy drugs are Actinomycin-D, Alkeran, Ara-C, Anastrozole, Asparaginase, BiCNU, Bicalutamide, Bleomycin, Busulfan, Capecitabine, Carboplatin, Carboplatinum, Carmustine, CCNU, Chlorambucil, Cisplatin, Cladribine, CPT-11, Cyclophosphamide, Cytarabine, Cytosine arabinoside, Cytoxan, Dacarbazine, Dactinomycin, Daunorubicin, Dexrazoxane, Docetaxel, Doxorubicin, DTIC, Epirubicin, Ethyleneimine, Etoposide, Floxuridine, Fludarabine, Fluorouracil, Flutamide, Fotemustine, Gemcitabine, Herceptin, Hexamethylamine, Hydroxyurea, Idarubicin, Ifosfamide, Irinotecan, Lomustine, Mechlorethamine, Melphalan, Mercaptopurine, Methotrexate, Mitomycin, Mitotane, Mitoxantrone, Oxaliplatin, Paclitaxel, Pamidronate, Pentostatin, Plicamycin, Procarbazine, Rituximab, Steroids, Streptozocin, STI-571, Streptozocin, Tamoxifen, Temozolomide, Teniposide, Tetrazine, Thioguanine, Thiotepa, Tomudex, Topotecan, Treosulphan, Trimetrexate, Vinblastine, Vincristine, Vindesine, Vinorelbine, VP-16, and Xeloda.

Useful cancer chemotherapy drugs also include alkylating agents such as Thiotepa and cyclosphosphamide; alkyl sulfonates such as Busulfan, Improsulfan and Piposulfan; aziridines such as Benzodopa, Carboquone, Meturedopa, and Uredopa; ethylenimines and methylamelamines including altretamine, triethylenemelamine, trietylenephosphoramide, triethylenethiophosphaoramide and trimethylolomelamine; nitrogen mustards such as Chlorambucil, Chlornaphazine, Cholophosphamide, Estramustine, Ifosfamide, mechlorethamine, mechlorethamine oxide hydrochloride, Melphalan, Novembiehin, Phenesterine, Prednimustine, Trofosfamide, uracil mustard; nitroureas such as Cannustine, Chlorozotocin, Fotemustine, Lomustine, Nimustine, and Ranimustine; antibiotics such as Aclacinomysins, Actinomycin, Authramycin, Azaserine, Bleomycins, Cactinomycin, Calicheamicin, Carabicin, Carminomycin, Carzinophilin, Chromoinycins, Dactinomycin, Daunorubicin, Detorubicin, 6-diazo-5-oxo-L-norleucine, Doxorubicin, Epirubicin, Esorubicin, Idambicin, Marcellomycin, Mitomycins, mycophenolic acid, Nogalamycin, Olivomycins, Peplomycin, Potfiromycin, Puromycin, Quelamycin, Rodorubicin, Streptonigrin, Streptozocin, Tubercidin, Ubenimex, Zinostatin, and Zorubicin; anti-metabolites such as Methotrexate and 5-fluorouracil (5-FU); folic acid analogues such as Denopterin, Methotrexate, Pteropterin, and Trimetrexate; purine analogs such as Fludarabine, 6-mercaptopurine, Thiamiprine, and Thioguanine; pyrimidine analogs such as Ancitabine, Azacitidine, 6-azauridine, Carmofur, Cytarabine, Dideoxyuridine, Doxifluridine, Enocitabine, Floxuridine, and 5-FU; androgens such as Calusterone, Dromostanolone Propionate, Epitiostanol, Rnepitiostane, and Testolactone; anti-adrenals such as aminoglutethimide, Mitotane, and Trilostane; folic acid replenisher such as frolinic acid; aceglatone; aldophosphamide glycoside; aminolevulinic acid; Amsacrine; Bestrabucil; Bisantrene; Edatraxate; Defofamine; Demecolcine; Diaziquone; Elfornithine; elliptinium acetate; Etoglucid; gallium nitrate; hydroxyurea; Lentinan; Lonidamine; Mitoguazone; Mitoxantrone; Mopidamol; Nitracrine; Pentostatin; Phenamet; Pirarubicin; podophyllinic acid; 2-ethyihydrazide; Procarbazine; PSK®; Razoxane; Sizofrran; Spirogermanium; tenuazonic acid; triaziquone; 2,2′,2″-trichlorotriethylamine; Urethan; Vindesine; Dacarbazine; Mannomustine; Mitobronitol; Mitolactol; Pipobroman; Gacytosine; Arabinoside (“Ara-C”); cyclophosphamide; thiotEPa; taxoids, e.g., Paclitaxel (TAXOL®, Bristol-Myers Squibb Oncology, Princeton, N.J.) and Doxetaxel (TAXOTERE®, Rhone-Poulenc Rorer, Antony, France); Chlorambucil; Gemcitabine; 6-thioguanine; Mercaptopurine; Methotrexate; platinum analogs such as Cisplatin and Carboplatin; Vinblastine; platinum; etoposide (VP-16); Ifosfamide; Mitomycin C; Mitoxantrone; Vincristine; Vinorelbine; Navelbine; Novantrone; Teniposide; Daunomycin; Aminopterin; Xeloda; Ibandronate; CPT-11; topoisomerase inhibitor RFS 2000; difluoromethylornithine (DMFO); retinoic acid; Esperamicins; Capecitabine; and pharmaceutically acceptable salts, acids or derivatives of any of the above. Also included are anti-hormonal agents that act to regulate or inhibit hormone action on tumors such as anti-estrogens including for example Tamoxifen, Raloxifene, aromatase inhibiting 4(5)-imidazoles, 4 Hydroxytamoxifen, Trioxifene, Keoxifene, Onapristone, And Toremifene (Fareston); and anti-androgens such as Flutamide, Nilutamide, Bicalutamide, Leuprolide, and Goserelin; and pharmaceutically acceptable salts, acids or derivatives of any of the above.

Cytokines can be also used as the therapeutic agent. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet growth factor; transforming growth factors (TGFs) such as TGF-α and TGF-β; insulin-like growth factor-I and -II; erythropoietin (EPO); osteoinductive factors; interferons such as interferon-α, -62 and -γ; colony stimulating factors (CSFs) such as macrophage-CSF (M-CSF); granulocyte-macrophage-CSF (GM-CSF); and granulocyte-CSF (GCSF); interleukins (ILs) such as IL-1, IL-1a, IL-2, IL-3, IL-4, IL-5, IL-6, IL-7, IL-8, IL-9, IL-11, IL-12, IL-15; a tumor necrosis factor such as TNF-α or TNF-β; and other polypeptide factors including LIF and kit ligand (KL). As used herein, the tern cytokine includes proteins from natural sources or from recombinant cell culture and biologically active equivalents of the native sequence cytokines.

The imaging agent can be any substance that can provide imaging information about a targeted site in a body of an animal such a mammal or a human being. The imaging agent can comprise magnetic material, such as iron oxide, for magnetic resonance imaging. For optical imaging, the active agent can be, for example, semiconductor nanocrystal or quantum dot. For optical coherence tomography imaging, the imaging agent can be metal, e.g. gold or silver, nanocage particles. The imaging agent can be also an ultrasound contrast agent such as a micro or nanobubble or iron oxide micro or nanoparticle.

### Compositions

The particles can be part of a composition such as a pharmaceutical composition. Such a composition can be a suspension comprising the selected particles described above for use in administering a therapeutic or imaging agent. To form the suspension, the particles can be suspended in an aqueous medium at a selected concentration. The optimal concentration will depend on the characteristics (e.g., solubilization properties) of the particle, type of therapeutic application and mode of administration. For example, compositions for oral administration can be relatively viscous, and may therefore contain a high concentration (e.g., >50%) of the particle. Solutions for bolus injections preferably contain a relatively concentrated suspension of the particles (e.g., 10-50%), but not so concentrated that it has an appreciably higher viscosity than saline (to minimize need for large-bore needles). The solution used for continuous intravenous infusion typically contains a relatively low concentration (e.g., 2-10% suspension) of the particles, due to the relatively large volumes of fluid that are administered.

The particles can be suspended in any suitable aqueous carrier vehicle. A suitable pharmaceutical carrier is one that is non-toxic to the recipient at the dosages and concentrations employed and is compatible with other ingredients in the formulation. Examples of suitable carrier vehicles include but are not limited to water, saline, Ringer's solution, dextrose solution, and 5% human serum albumin. Suspensions for use in injectable formulations are preferably isotonic with the subject's blood. Generally, the carrier can contain minor amounts of additives such as substances that enhance isotonicity and chemical stability, e.g., buffers and preservatives, as well as low molecular weight (less than about 10 residues) polypeptides, proteins, amino acids, carbohydrates including glucose or dextrans, chelating agents such as EDTA, or other excipients.

Prior to administration to a subject, the suspension of particles can be sterilized by a suitable sterilization method. Particles fabricated from a heat-stable material can be heat-sterilized, e.g., using an autoclave. Particles fabricated from a not heat-stable material may be sterilized by passage through a commercially-available sterilization filter, e.g., a 0.2 μm filter. Of course, filtration may be used only in cases where the particles is smaller than the pores of the sterilizing filter.

The particles can be administered to a subject in need of therapeutic intervention via any suitable administration method. The particular method employed for a specific application is determined by the attending physician. The particles can be administered by one of the following routes: topical, parenteral, inhalation, oral, vaginal and anal. Intravascular administration, which includes intravenous (i.v.), intramuscular (i.m.) and subcutaneous (s.c.) injection, may be particularly preferred.

Intravascular administration can be either local or systemic. Local intravascular delivery can be used to bring the particles in the vicinity of a body site having a known tumor or inflammation by use of guided catheter system, such as a CAT-scan guided catheter. General injection, such as a bolus i.v. injection or continuous/trickle-feed i.v. infusion are typically systemic.

The selected particles are injected into the blood stream and allowed to circulate and localize to their target site. Preferably, the selected particles are injected to a vasculature of a body site for which the particles are selected to provide enhanced delivery into fenestrated blood vessels of the body site.

Although the foregoing description refers to particular preferred embodiments, it will be understood that the present invention is not so limited. It will occur to those of ordinary skill in the art that various modifications may be made to the disclosed embodiments and that such modifications are intended to be within the scope of the present invention. All of the publications, patent applications and patents cited in this specification are incorporated herein by reference to the extent that they describe materials, methods and other details that are supplementary to the disclosure herein.

What is claimed is:

1. A method for targeting a fenestrated blood vessel in a body site, comprising administering to a subject in need thereof a composition comprising particles having a radius that permits enhanced delivery into the fenestrated blood vessel of the body site, wherein the particles comprise at least one active agent.

2. The method of claim 1, wherein the at least one active agent comprises a therapeutic agent.

3. The method of claim 2, wherein the therapeutic agent is an anticancer agent.

4. The method of claim 1, wherein the active agent comprises an imaging agent.

5. The method of claim 1, wherein the particles comprise a nanoporous material.

6. The method of claim 5, wherein the nanoporous material is a nanoporous silicon.

7. The method of claim 5, wherein the nanoporous material is a nanoporous oxide material.

8. The method of claim 7, wherein the nanoporous oxide material is a nanoporous silicon dioxide.

9. The method of claim 1, wherein one or more of said particles comprise a biodegradable material.

10. The method of claim 1, wherein one or more of said particles comprise at least one recognition moiety disposed on a surface of each of the particles.

11. The method of claim 10, wherein the at least one recognition moiety comprises a renormalized vasculature recognition moiety.

12. The method of claim 10, wherein the at least one recognition moiety comprises a coopted vasculature recognition moiety.

13. The method of claim 10, wherein the at least one recognition moiety comprises an angiogenesis vasculature recognition moiety.

14. The method of claim 10, wherein the at least one recognition moiety comprises hydrophilic polymer chains.

15. The method of claim 1, wherein one or more of said particles are selected from the group consisting of liposomes, fullerene nanoparticles, semiconductor nanoparticles and metal nanoparticles.

16. The method of claim 1, further comprising fabricating the one or more particles.

17. The method of claim 15, wherein the fabricating comprises fabricating by a top-down technique.

18. The method of claim 1, wherein the administering comprises injecting the composition intravascularly.

19. The method of claim 18, wherein the injecting comprises injecting the composition in a vasculature of the body site.

20. The method of claim 1, wherein the subject is a mammal.

21. The method of claim 20, wherein the subject is a human.

22. The method of claim 1, wherein the body site is selected from the group consisting of brain, skin, skeletal muscle, lung, heart, kidney, stomach and intestine.

23. The method of claim 1, wherein the composition comprises a suspension of the particles.

24. The method of claim 1, wherein a condition responsible for the fenestrated blood vessel condition is a tumor.

25. The method of claim 1, further comprising administering to the subject a vasculature normalizing agent.

26. A method of improving efficacy of a composition comprising particles that comprise at least one active agent, the method comprising
selecting particles from a first population of micro or nanoparticles, such that the particles have a radius that permits enhanced delivery into fenestrated blood vessels of a target body site and
forming a composition comprising the selected particles.

27. The method of claim 26, wherein the selected particles constitute at least 10% of the first particles by number.

28. The method of claim 27, wherein the selected particles constitute at least 20% of the first particles by number.

29. The method of claim 28, wherein the selected particles constitute at least 50% of the first particles by number.

30. The method of claim 29, wherein the selected particles constitute at least 80% of the first particles by number.
